(19) United States
(12) Patent Application Publication
(10) Pub. No.: US 2024/0226146 A9
(43) Pub. Date: Jul. 11, 2024

(54) MEDICAL DEVICE FOR THE REPAIR OF A SPINAL OR NERVE LESION, AND SURGICAL METHOD

(71) Applicant: Michele CONTI, Nogaredo (Trento) (IT)

(72) Inventor: Michele CONTI, Nogaredo (Trento) (IT)

(21) Appl. No.: 18/102,598

(22) Filed: Jan. 27, 2023

(30) Foreign Application Priority Data
Oct. 19, 2022 (IT) 102022000021567

(51) Int. Cl. A61K 35/12; A61L 27/38

(52) U.S. Cl. CPC A61K 35/12; A61L 27/3804; A61L 27/3834

(57) ABSTRACT

A medical device for repairing a lesion in a spinal cord or in a peripheral nerve is provided. The medical device has a flexible support made of expanded polytetrafluoroethylene. Stem cells suitable for being oriented along a first growth direction or a second growth direction are at least partially embedded on the flexible support that is suitable for taking an extended configuration and a wound configuration. In the wound configuration, the flexible support is suitable for being wound around the spinal cord so that the first and second growth directions are substantially statistically parallel to a neuronal extension direction of neurons of the spinal cord. Surgical methods of treating a spinal injury involving using the medical device are also provided.

### CROSS-REFERENCE TO RELATED APPLICATION

This application claims priority to Italian Patent Application No. 102022000021567 filed on Oct. 19, 2022, the contents of which are incorporated by reference in their entirety.

### FIELD OF THE INVENTION

The present invention relates to a medical device for repairing a spinal or nerve lesion, of the spinal cord or of a peripheral nerve, respectively, and a surgical method for repairing a spinal lesion.

### BACKGROUND OF THE INVENTION

It is known that neuropathologies involve a degeneration and damage to the nervous system, in particular of the neurons in the brain, spinal cord and peripheral nervous system. A damage or alteration of the neural network leads to the creation of anomalous structures and functions with a drastic reduction in the quality of life. As regards damage to the neural network of the spine (known as Spine Cord Injury), the need is felt to explore new therapeutic strategies to treat this pathology. Damage to the spinal cord is related to two types of lesions: 1) primary lesions that concern physical damage immediately following the traumatic event, such as lacerations, bruises, compressions and contractions of the neuronal tissue; and 2) secondary lesions resulting from the primary lesions that may affect long-term mobility. The secondary lesions are characterized by inflammatory processes, alterations of the local ion concentration, loss of the local vascular system with consequent decrease in blood flow and penetration of serum proteins into the spinal cord. These changes lead to demyelization, ischemia, necrosis, and apoptosis of spinal cord neuronal tissue.

Current strategies developed have led to therapies that target primary, secondary, or both lesions; the main goal is to block the cascading mechanisms in place during secondary lesions. In particular, a large variety of therapies have been developed to alter the neuroinflammatory process, reduce free radical damage, improve blood flow and counteract the effects of local ionic changes. All therapies that target secondary lesions are based on the use of drugs or active molecules (e.g. myelin, associated with glycoproteins or inhibitors).

Within this scenario, with the numerous clinical studies carried out, only the therapy with the glucocorticoid methylprednisolone (MP) seems to be effective, albeit to a limited extent and with limited improvements in the treated patients.

Thanks to new therapeutic methodologies and the discovery of new nanotechnological applications, part of the research is currently focused on a regenerative as well as therapeutic approach, in particular aimed at the regeneration of axons.

In particular, attempts have been made to promote neuronal regrowth by directly applying autologous stem cells in proximity to the neuronal lesion, without however obtaining effective results.

The applicant has designed a neural repair device for spinal cord and nerves, described in EP3843800A1, suitable for promoting the growth and differentiation of stem cells on lesions of peripheral, central or spinal cord nerves.

Such device wraps around the damaged spinal cord area, acting as a support for the stem cells that are applied directly to the lesion. The device comprises a support and a binding layer with polyamides or polylactates which houses nanotubes suitable for promoting the orientation of the stem cells in the required regeneration direction.

However, this device has some drawbacks. In fact, the risk of toxicity associated with the use of such a device is contained, but not excluded, and this risk must be mitigated by means of specific manufacturing expedients.

In fact, one embodiment of this device also requires the use of a second flexible support which isolates the patient's biological tissue from these nanoparticles, to reduce the risk of toxicity in the subject in which this medical device is implanted.

Therefore, the medical device described in the cited document requires a particular construction and implies specific measures to reduce the risk of toxicity associated with the use of nanoparticles.

Furthermore, the regeneration of the neural connections at the medulla level by means of the device devised by the Applicant takes place in an effective but not optimized manner.

### SUMMARY OF THE INVENTION

A strong need is thus felt for a medical device and a surgical method that are able to improve and promote neuronal regeneration on a spinal cord lesion.

In particular, the aim of the present invention is to provide a medical device that overcomes the drawbacks of the prior art and in particular of the device described in EP3843800A1 and that is capable of improving the repair of a spinal or nerve lesion.

Furthermore, the aim of the present invention is to provide a medical device which is simpler to produce and which further reduces the risk of toxicity associated with the use of a neuronal regeneration device.

A further objective of the present invention is to provide a surgical method which allows spinal regeneration to be made more efficient and rapid and which promotes the restoration of nerve connections.

According to the present invention, the aforementioned objectives are achieved by a medical device and by a surgical method according to the appended independent claims. Preferred embodiments of the present invention are defined in the dependent claims.

According to a further aspect, the aforementioned objectives are also achieved by a surgical method for repairing a spinal lesion by means of a medical device according to the present invention.

The dependent claims describe preferred or advantageous embodiments of the present invention, comprising further advantageous features.

### BRIEF DESCRIPTION OF THE DRAWINGS

The features and advantages of the medical device and of the surgical method will become apparent from the following description of a number of preferred embodiments, given by way of non-limiting example, with reference to the attached figures, in which:

FIG. 1 shows a schematic cross-sectional view of a spinal cord;

FIG. 2a shows a schematic view of a healthy spinal cord with reference to a lateral section of a vertebral column, in which the ascending and descending connections of the nerve connections are visible;

FIG. 2b shows a schematic lateral sectional view of a damaged spinal cord, with reference to a section through a vertebral column, in which an interruption of the ascending and descending nerve connections is shown;

FIG. 3 schematically shows a perspective view of a medical device according to an embodiment of the present invention, in a wound configuration, in a winding step;

FIG. 4 schematically shows a planar view of the medical device of FIG. 3 in extended configuration;

FIG. 5 shows a cross-sectional view of a medical device according to an embodiment of the present invention wound around the spinal cord, in which a dot indicates a cranial-caudal direction, while a cross indicates a caudal-cranial direction;

FIGS. 6 to 10 show the steps of a surgical method for repairing a spinal lesion, and in particular:

FIG. 6 shows a schematic front elevation view of a region of a damaged spinal cord, in which, for each side—right and left—of the spinal cord, the upper and lower spinal roots of the spinal lesion are visible;

FIG. 7 shows the same schematic front elevation view of FIG. 6, in which the upper and lower spinal roots, on each side, have been severed and connected to each other to generate a bypass connection;

FIG. 8 shows the same schematic front elevation view of FIG. 7, in a step of winding of the spinal cord at the spinal lesion, by means of a medical device according to an embodiment of the present invention;

FIG. 9 shows the same schematic front elevation view of FIG. 8, in a step of winding of the spinal roots at the respective bypass connecting region, by means of respective additional medical devices, according to an embodiment of the present invention; and

FIG. 10 shows a schematic view of a cross section of a spinal cord regenerated by the surgical method according to an embodiment of the present invention, in parallel with a schematic elevation view thereof, in which the restoration of the neural pathways is shown.

### DETAILED DESCRIPTION

With reference to the above figures, reference numeral 1 indicates as a whole a medical device for repairing a lesion 200, spinal or nerve, respectively, in a spinal cord 2 or in a peripheral nerve.

According to the present invention, the medical device 1 for repairing a lesion 200 in the spinal cord 2 or in a peripheral nerve comprises a biocompatible flexible support 10 made of expanded polytetrafluoroethylene (ePTFE).

In one embodiment, an inner surface 120 and an outer surface 150, opposite to the inner surface 120, may be identified on the flexible support 10.

Preferably, the flexible support 10 is a strip of expanded polytetrafluoroethylene (ePTFE).

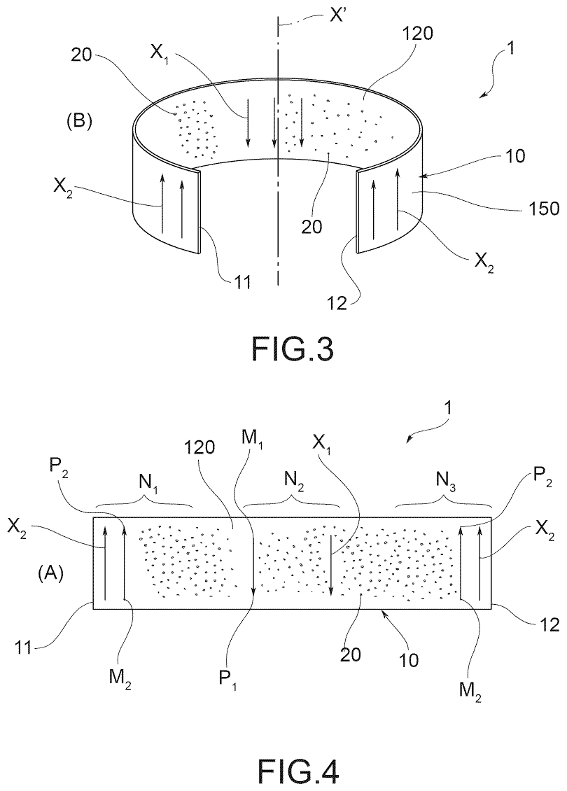

According to the present invention, as shown for example in FIG. 3, stem cells 20 are at least partially embedded on the inner surface 120 of the flexible support 10, suitable for being oriented along a first growth direction X1 or along a second growth direction X2.

The term “oriented” means that stem cells are suitable for being guided in their differentiation in the first growth direction X1 or in the second growth direction X2.

According to the present invention, the flexible support 10 is suitable for taking an extended configuration A and a wound configuration B. In the wound configuration B, shown for example in FIG. 3, the flexible support 10 is suitable for being wound around the spinal cord 2 and/or a spinal root or a peripheral nerve in such a way that, when the device is wound around the spinal cord 2 and/or the spinal root or the peripheral nerve, the inner surface 120 faces the spinal cord 2, towards the spinal root or towards the peripheral nerve, and the first and second growth directions X1, X2 are substantially statistically parallel to the neuronal extension direction X′ of the spinal cord and/or of the spinal root or of the peripheral nerve neurons.

In the present discussion, with the term “statistically” it may be understood that in a statistical way there is a predominance of growth of stem cells in one direction (for example in the first growth direction X1), or that the first growth direction X1 is substantially parallel to a statistically predominant extension direction of neurons in the spinal cord.

Preferably, in the extended configuration A, shown for example in FIG. 4, the flexible support has the shape of a thin planar strip. Preferably, in the wound configuration B, the flexible support has the shape of a tube, i.e. an annular shape, which winds around the spinal cord or a peripheral nerve.

In particular, before being implanted, the biocompatible flexible support 10 is preferably in an extended configuration A, while, once the implant has been performed, the device is arranged in a wound configuration B around the spinal cord and/or the spinal root or the peripheral nerve. It is clear that with extended configuration A it is therefore meant a generic configuration of the flexible support in the pre-implantation step, different from the configuration wound around the spinal cord. In case the flexible support is a thin strip, the extended configuration A is a planar configuration, while in the wound configuration B, the thin strip takes an annular shape around the spinal cord and/or the spinal root or the peripheral nerve.

Preferably, in a winding step, shown for example in FIG. 3, the flexible support 10 takes a “C” cross-section and is suitable for being sewn to form a tube around the spinal cord 2 and/or the spinal root or the peripheral nerve.

Preferably, the stem cells 20 are not cells of embryonic origin. Even more preferably, the stem cells 20 are mesenchymal cells of adipose origin.

In one embodiment, the stem cells 20 comprise mesenchymal cells from the umbilical cord. This embodiment is particularly advantageous for use of the medical device according to the present invention on new-born individuals with disabilities.

In an advantageous embodiment, the stem cells 20 comprise autologous stem cells of a subject in which the medical device 1 is implantable, obtained from the adipose tissue of the subject, i.e., adipose tissue derived stem cells (ADSCs).

Preferably, the stem cells 20 are embedded directly on the flexible support 10, i.e., without any interposed layer.

That is, the flexible support 10 lacks a binding layer, and/or additional nanoparticles.

Advantageously, therefore, the medical device is simple to manufacture. Furthermore, providing a medical device completely free of binding layers and above all of nanoparticles further reduces the risk of toxicity of the device on the spinal cord, on the spinal roots or on the nerve compared to devices of the prior art which provide for the use of nanoparticles.

Furthermore, advantageously, the applicant has surprisingly found that expanded polytetrafluoroethylene (ePTFE) is efficient for use in the treatment of neural lesions, as well as for the common use at the vascular level.

In an advantageous embodiment, the medical device 1 comprises one or more growth factors deposited on the flexible support 10 so as to promote the directionality and proliferation of the stem cells 20 along the first growth direction X1 or the second growth direction X2.

Preferably, the first and the second growth directions X1, X2 each extend from a respective starting point M1, M2 toward a respective arrival point P1, P2, and the one or more growth factors are deposited in correspondence of each respective starting point M1, M2 so that the extension of the stem cells 20 from the respective starting point M1, M2 toward the respective arrival point P1, P2 is promoted.

Preferably, the first growth direction X1 is the efferent (or cranial-caudal) direction of the neurons and the second growth direction X2 is the afferent (or caudal-cranial) direction of the neurons.

Preferably, therefore, in the case of a thin flexible support 10, functionalized regions, containing stem cells 20 suitable for being oriented in the first growth direction X1 or in the second growth direction X2 are arranged in succession and adjacent to each other on the flexible support.

For example, with reference to the example of FIG. 4, the medical device 1 comprises a first region N1, close to a left end 11 of the flexible support 10, in which the growth factor is deposited in such a way that the functionalization and proliferation of the stem cells in the first region N1 is promoted along the second growth direction X2. Furthermore, for example, the medical device 1 comprises a second region N2, adjacent to the first region N1 and arranged more towards the center of the biocompatible flexible support 10, in which the growth factor is deposited in such a way that the functionalization and proliferation of stem cells in the second region N2 is promoted along the first growth direction X1, opposite to the second growth direction X2.

Furthermore, the medical device 1 preferably comprises a third region N3, close to a right end 12 of the biocompatible flexible support 10, in which the growth factor is deposited in such a way that the functionalization and proliferation of the stem cells in the third region N3 is promoted along a third growth direction, preferably again along the second growth direction X2.

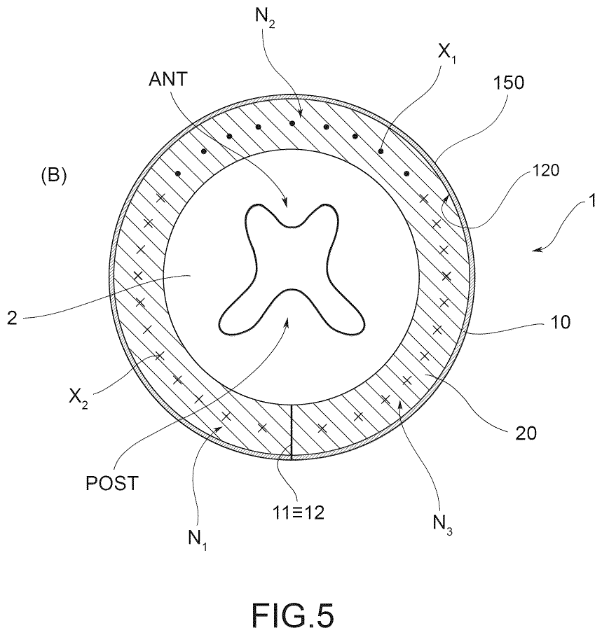

In this embodiment variant, as shown for example in FIG. 5, when the medical device is implanted around the spinal cord in the wound configuration B (i.e. with the two ends 11 and 12 of the flexible support joined together), the second region N2 is adjacent to the front region ANT of the spinal cord, so that the differentiation of stem cells in the first growth direction X1, i.e. in the cranial-caudal (or efferent) direction, is promoted while the first and third regions N1, N3 are adjacent to the rear region POST of the spinal cord, so that the differentiation of stem cells in the second growth direction X2, i.e. in the caudal-cranial (or afferent) direction, is promoted.

In FIG. 5, to facilitate the intelligibility of the present invention, the layer of stem cells 20 deposited directly on the flexible support 10 has been deliberately represented in an exaggerated manner, and therefore clearly not to scale with respect to the thickness of the flexible support 10.

In one embodiment, the outer surface 150 is at least partially coated with a reinforcement coating. The outer surface 150 is suitable for facing away from the spinal cord 2 when the medical device 1 is in the wound configuration B.

Preferably, the reinforcement coat is a heparin film.

It is an object of the present invention also a method for manufacturing a medical device 1 according to an embodiment of the present invention, comprising the steps of
providing a flexible support 10 made of expanded polytetrafluoroethylene;
arranging stem cells 20 on an inner surface 120 of the flexible support 10; and
depositing a growth factor on the inner surface 120 to promote the development of the stem cells 20 along a first growth direction X1 or a second growth direction X2.

In an advantageous embodiment, the stem cells 20 are mesenchymal cells from adipose tissue and are obtained by the following steps:
collecting autologous adipose tissue from the subject in which the medical device 1 is suitable for being implanted;
filtering the adipose tissue through a semipermeable membrane, so as to isolate the mesenchymal cells from blood and/or oily residues;
concentrating and injecting the mesenchymal cells on the flexible support 10.

In one embodiment, the manufacturing method further comprises a step of expanding the stem cells with mesenchymal cells from autologous umbilical cord.

This embodiment is particularly useful and effective for the treatment of spinal lesions in new-born individuals.

It forms an object of the present invention also a surgical method for repairing a spinal lesion 200 on a damaged spinal cord 2, comprising the following steps:
a) providing a medical device 1 according to the present invention or manufacturing a medical device 1 by means of the manufacturing method according to the present invention;
b) winding the spinal cord 2 by means of the medical device 1 in correspondence of the spinal lesion 200.

Preferably, the steps of such a method are performed sequentially as listed.

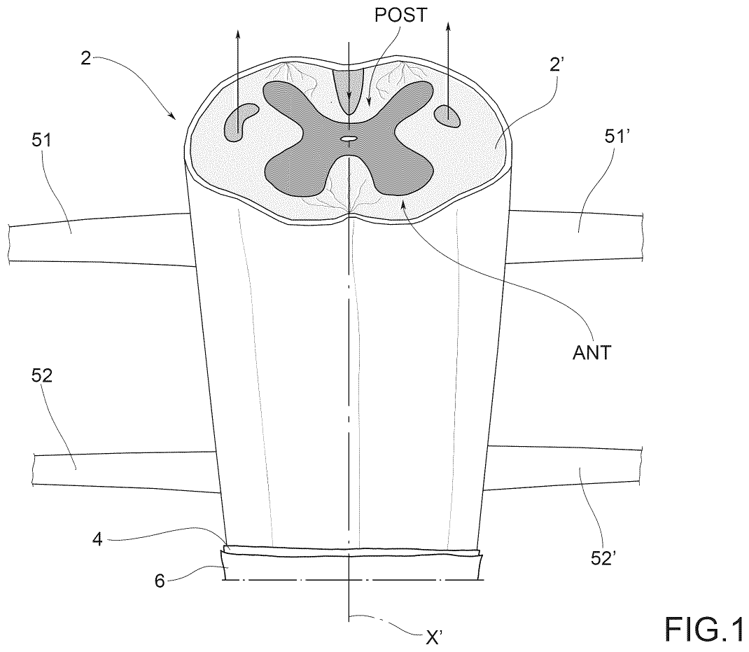

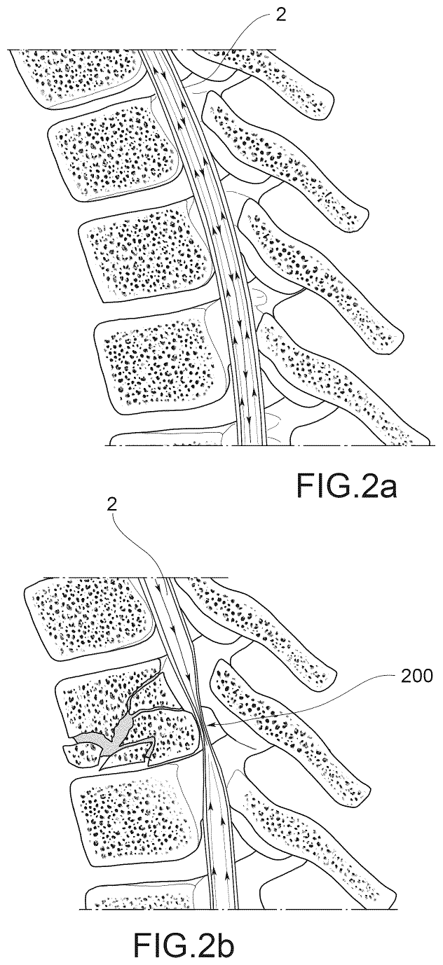

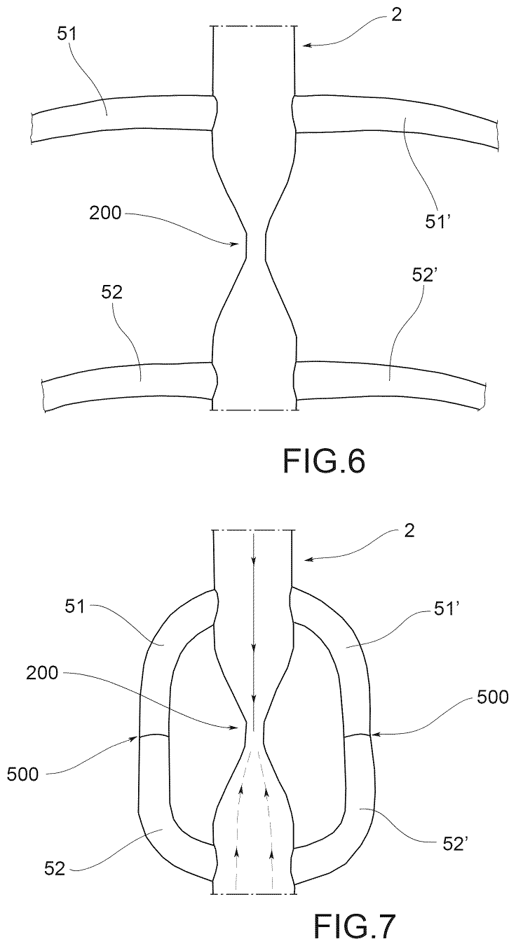

In a preferred embodiment, with reference to the accompanying FIGS. 1 and 6, an upper spinal root 51 and a lower spinal root 52 are identified at the spinal lesion 200 which branch off from the spinal cord 2 on the same first side, left or right, with respect to the spinal cord 2, above and below the spinal lesion 200, respectively.

The object of the present invention is a surgical method for repairing a spinal lesion 200 on a damaged spinal cord 2, which comprises the following steps:
a) providing a first medical device 1 comprising a biocompatible flexible support 10 and stem cells 20;
a1) providing a further medical device 1 comprising a flexible support 10 and stem cells 20,
b) winding the spinal cord 2 by means of the medical device 1 at the spinal lesion 200,
c) carrying out a bypass operation comprising the following steps:
c1) cutting the upper spinal root 51;
c2) cutting the lower spinal root 52;
c3) making an end-to-end connection between said severed upper spinal root 51 and lower spinal root 52, in correspondence of a bypass connecting region 500;
c4) in correspondence of the bypass connecting region 500, winding the connected upper 51 and lower 52 spinal roots, by means of the further medical device 1.

Preferably, steps a) to c4) are performed in the order as listed.

It is clear that this method is applicable to any medical device 1 comprising a flexible biocompatible support 10 and stem cells 20. Preferably, but not exclusively, the medical device 1 is a medical device 1 according to the present invention.

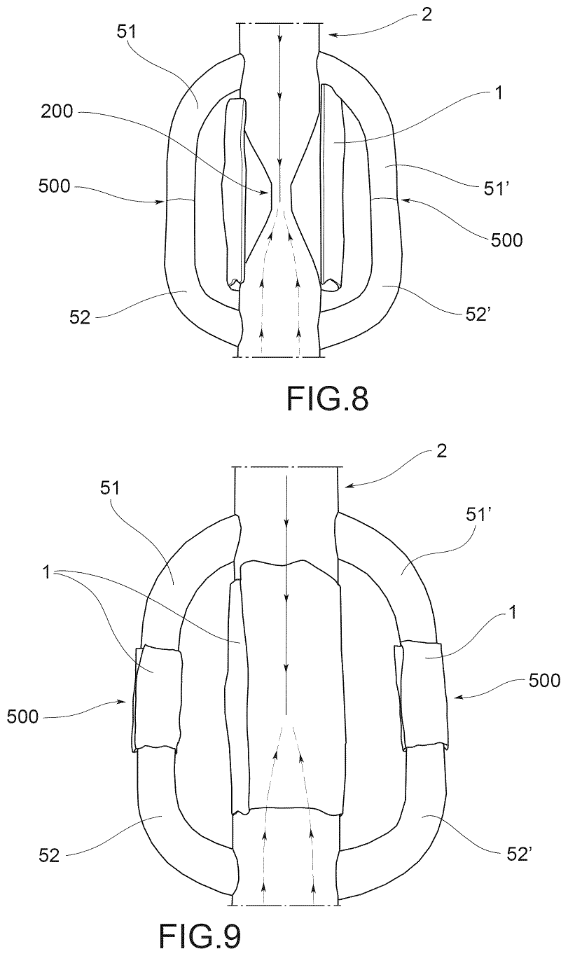

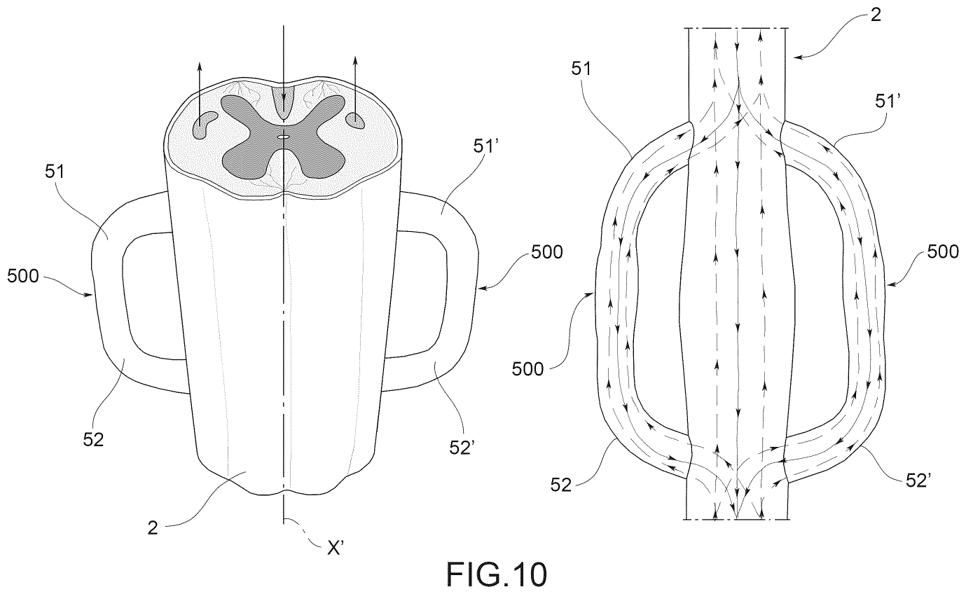

Preferably, the steps of the surgical method are performed in sequence as listed and are represented in the accompanying FIGS. 6 to 10.

In one embodiment, with reference to the accompanying FIGS. 1 and 6, a second upper spinal root 51′ and a second lower spinal root 52′ are identified at the spinal lesion 200 which branch off from the spinal cord 2 on the same second side, right or left, opposite the first side, above and below the spinal lesion 200, respectively.

In one embodiment, the surgical method provides for performing steps a1), c1), c2), c3) and c4) also on the second upper 51′ and lower 52′ spinal root, i.e. performing a bypass operation on both sides of the spinal cord 2.

Preferably, also the steps from a1) to c4) applied on the second upper 51′ and lower 52′ spinal root are performed in the order as listed.

Preferably, the upper 51, 51′ and lower 52, 52′ spinal roots are dorsal roots, or lumbar roots, or cervical roots.

In a preferred embodiment, the severing of the spinal roots by means of steps c1) and c2) is performed cleanly, without tearing and without bipolar coagulation. In this way, neural regeneration is further stimulated and the connection between the roots at the bypass connecting region is automatically oriented correctly, so that the front region of a root matches the front region of the root connected thereto.

The object of the present invention is a surgical method for repairing a spinal lesion 200 on a damaged spinal cord 2, comprising the following steps:
m) providing a medical device 1 comprising a flexible support 10 and stem cells 20;
n) identifying an upper spinal root 51 and a lower spinal root 52 which branch off from the spinal cord 2 on the same first side, left or right, with respect to the spinal cord 2, above and below the spinal lesion 200, respectively;
o) carrying out a bypass operation comprising the following steps:
o1) cutting the upper spinal root 51;
o2) cutting the lower spinal root 52;
o3) making an end-to-end connection between said severed upper spinal root 51 and the lower spinal root 52, at a bypass connecting region 500;
o4) at the bypass connecting region 500, winding the connected upper 51 and lower 52 spinal roots, by means of the medical device 1.

In one embodiment, the surgical method provides for a step m′) of providing a further medical device 1 comprising a flexible support 10 and stem cells 20 and a step n′) of identifying a second upper spinal root 51′ and a second lower spinal root 52′ branching off from the spinal cord 2 from the same second side, right or left, opposite to the first side, above and below the spinal lesion 200, respectively.

In one embodiment, the surgical method provides for performing steps o1), o2), o3) and o4) also on the second upper 51′ and lower 52′ spinal root, i.e. performing a bypass operation according to step o) on both sides of the spinal cord 2.

That is, according to one aspect of the present invention, the regeneration of the neural connections at the spinal lesion 200 takes place exclusively by means of a surgical method which employs a medical device 1 exclusively at the spinal root bypass regions 51, 51′, 52, 52′ and does not involve the winding of the spinal cord itself.

Preferably, steps m) to o4) and steps m′) to o4) are performed in the order as listed.

In a preferred embodiment, the severing of the spinal roots by means of steps c1), c2) or steps o1) and o2) is performed cleanly, without tearing and without bipolar coagulation. That is, steps c1), c2) or steps o1), 02) provide for cutting the spinal roots 51, 52, 51′, 52′ cleanly, without tearing and without bipolar coagulation.

Preferably, the steps of the surgical method are performed in sequence as listed and are represented in the accompanying FIGS. 6 to 10.

In a particularly advantageous embodiment, the medical device 1 and/or the further medical device 1 used in the methods described is a medical device 1 according to an embodiment of the present invention or is made by means of an embodiment of the manufacturing method according to the present invention.

As is known, with reference to FIG. 1, the spinal cord 2 comprises an outer layer of dura mater, or dural sac 6, an inner layer of pia mater 2′ and an intermediate layer of arachnoid 4 interposed between the inner layer of pia mater 2′ and the dural sac 6.

In one embodiment, the method comprises, before step b), a step of cutting the dural sac 6 and the arachnoid 4, through which direct access to the pia mater 2′ is allowed, from the outside.

Furthermore, a method for implanting the medical device comprises the steps of:
performing a dorsal laminectomy in the vicinity of a spinal lesion 200 to expose the dural sac 6;
opening the dural sac 6 and the arachnoid 4 and exposing the pia mater 2′;
possible anchoring of the dural flaps to the muscular walls;
possible resection of the denticulate ligaments to allow easier access;
inserting the medical device 1 through the surgical method according to the present invention, in such a way that it is completely wound around the spinal cord, for example like a shirt collar around the neck.

In one embodiment, a method for implanting the medical device comprises the following steps:
incising the epispinous midline of the skin;
skeletonization of the paravertebral lodges;
removing spinous processes and laminae with spinolaminectomy; and
opening of the yellow ligament and exposure of the dural sac.

Preferably, the insertion of the medical device 1 takes place by sliding one end 11 or 12 of the flexible support 10 starting from one side of the cord towards the front region ANT until it re-emerges from the opposite side of the cord.

Once this operation has been performed, the two ends 11 and 12 of the flexible support are then joined, for example by suturing near the rear region POST of the spinal cord, so that the device is completely wound around the spinal cord.

One then proceeds to the known steps of closing the dural sac and the remaining tissues of the access opening.

Preferably, the steps of the implantation method are performed in the order as listed.

It forms an object of the present invention a use of a medical device 1 according to an embodiment of the present invention for the treatment of a peripheral or spinal nerve.

It forms an object of the present invention a use of expanded polytetrafluoroethylene (ePTFE) for manufacturing a flexible support for the treatment of a peripheral or spinal nerve.

Preferably, the flexible support is suitable for being used for manufacturing a medical device 1 according to an embodiment of the present invention.

It is an object of the present invention a use of expanded polytetrafluoroethylene (ePTFE) and stem cells 20 for manufacturing a medical device for the treatment of a peripheral or spinal nerve.

Preferably, the expanded polytetrafluoroethylene is suitable for manufacturing a flexible support 10 as described in the present application and the stem cells 20 are suitable for being embedded directly in the flexible support 10 to manufacture a medical device 1 according to an embodiment of the present invention.

Innovatively, the present invention overcomes the drawbacks of the prior art.

Advantageously, the medical device is suitable for improving and promoting neuronal regeneration in the vicinity of a spinal or nerve lesion.

In particular, the medical device according to the present invention provides support to the growth of the stem cells, guiding the correct growth direction. Furthermore, due to the fact that the support is made of ePTFE, the medical device is extremely flexible and makes implantation and winding around the medulla and/or spinal roots easier.

Advantageously, the medical device according to the present invention is simple to manufacture and is obtained starting from a material already available on the market, such as ePTFE, tested for its non-toxicity on the human body and surprisingly effective for neural regeneration.

Advantageously, the surgical method according to the present invention is suitable for effectively restoring the neural connections.

Advantageously, providing a surgical method that performs a bypass operation on one side of a spinal lesion, with or without winding of the spinal cord at the spinal lesion, allows a mid-spinal lesion to be effectively treated.

According to a further advantage, the surgical method in the embodiment which provides for performing a bypass operation on both spinal roots, acts on two or three connecting points (right spinal root and left spinal root, and optionally the spinal cord) and allows for an even more efficient restoration of electrical conduction at the synaptic level.

In fact, according to a fundamental advantage, by exploiting three nerve connections, rather than just the connection of the spinal cord itself, the resistivity of the entire system is reduced. This clearly happens because the three connections work in parallel, and consequently, the total resistance of the system is lower than that of the single connection. Since the resistance is inversely proportional to the intensity of the current, it follows that the conductivity of the system itself has increased.

Therefore, according to a particularly advantageous aspect, the surgical method allows the neural conduction of the nervous system to be increased, thus optimizing the efficiency of the neural regeneration process of the ascending and descending nervous connections at the level of the cord.

Conveniently, the applicant has demonstrated also experimentally, by means of tests carried out on cadavers, that the method according to the present invention allows the electrical signal conducted through the nerve roots to be improved, improving the regeneration mechanism of the ascending and descending connections at the level of the spinal cord.

According to a further advantage, the surgical method is simple to implement for experts in the field and allows the thoracic roots to be exploited to implement the efficiency of spinal cord regeneration, without causing any damage to the patient.

It is clear that, to the embodiments of the device for repairing a spinal or nerve lesion, of the manufacturing method thereof, and of the surgical method, those skilled in the art, in order to satisfy specific needs, may make variants or substitutions of elements with others functionally equivalent.

These variants are also contained within the scope of protection as defined by the following claims. Moreover, each variant described as belonging to a possible embodiment may be implemented independently of the other variants described.

What is claimed is:

1. A medical device for repairing a lesion in a spinal cord or a peripheral nerve, comprising a biocompatible flexible support suitable for taking an extended configuration and a wound configuration and comprising stem cells suitable for being oriented along a first growth direction or a second growth direction,
wherein, in said wound configuration, the biocompatible flexible support is windable around the spinal cord and/or a spinal root, or the peripheral nerve, so that said first and second growth directions are substantially statistically parallel to a neuronal extension direction of neurons of the spinal cord or of the peripheral nerve,
wherein said biocompatible flexible support comprises an inner surface such that when the medical device is wound around said spinal cord and/or the spinal root or the peripheral nerve, the inner surface faces the spinal cord and/or the spinal root or the peripheral nerve, and
wherein the biocompatible flexible support is made of expanded polytetrafluoroethylene (ePTFE) and the stem cells are at least partially embedded on the inner surface of said biocompatible flexible support.

2. The medical device of claim 1, wherein said stem cells comprise autologous stem cells of a subject in which the medical device is implantable, obtained from adipose tissue of the subject, namely adipose tissue derived stem cells (ADSCs).

3. The medical device of claim 1, wherein the stem cells are embedded directly on the biocompatible flexible support without any interposed layer.

4. The medical device of claim 1, comprising one or more growth factors deposited on the biocompatible flexible support to promote directionality and proliferation of the stem cells along said first growth direction or said second growth direction.

5. The medical device of claim 4, wherein said first and second growth directions each extend from a respective starting point toward a respective arrival point, and wherein said one or more growth factors are deposited in correspondence of each respective starting point so that extension of said stem cells from the respective starting point toward the respective arrival point is promoted.

6. The medical device of claim 1, wherein the first growth direction is an efferent or cranial-caudal direction of the neurons and the second growth direction is an afferent or caudal-cranial direction of the neurons.

7. The medical device of claim 1, wherein the biocompatible flexible support comprises an outer surface at least partially coated with a reinforcement coating, said outer surface being suitable for facing away from the spinal cord when the medical device is in the wound configuration.

8. A surgical method for repairing a lesion in a spinal cord, wherein an upper spinal root and a lower spinal root are identified in correspondence of the lesion in the spinal cord which branch off from the spinal cord on a same first side, left or right, with respect to the spinal cord, above and below the lesion in the spinal cord, respectively,
the surgical method comprising:
a) providing a medical device comprising a biocompatible flexible support and stem cells;
a1) providing a further medical device comprising a biocompatible flexible support and stem cells,
b) winding said spinal cord by the medical device at the lesion in the spinal cord,
c) carrying out a bypass operation comprising:
c1) cutting the upper spinal root;
c2) cutting the lower spinal root;
c3) making an end-to-end connection between said severed upper and lower spinal roots, at a bypass connecting region; and
c4) at said bypass connecting region, winding said connected upper and lower spinal roots, by said further medical device.

9. The surgical method of claim 8, wherein a second upper spinal root and a second lower spinal root are identified at the lesion in the spinal cord which branch off from the spinal cord on a same second side, right or left, opposite the first side, above and below the lesion in the spinal cord, respectively,
the surgical method comprising performing steps a1), c1), c2), c3) and c4) also on said second upper and lower spinal roots, that is performing a bypass operation on both sides of the spinal cord.

10. A surgical method for repairing a lesion in a spinal cord, comprising:
m) providing a medical device comprising a biocompatible flexible support and stem cells;
n) identifying an upper spinal root and a lower spinal root branching off from the spinal cord on a same first side, left or right, with respect to the spinal cord, above and below the lesion in the spinal cord, respectively;
o) carrying out a bypass operation comprising:
o1) cutting the upper spinal root;
o2. cutting the lower spinal root;
o3) making an end-to-end connection between said severed upper and lower spinal roots, in correspondence of a bypass connecting region; and
o4) in correspondence of said bypass connecting region, winding said connected upper and lower spinal roots, by said medical device.

11. The surgical method of claim 10, comprising:
m′) providing a further medical device comprising a biocompatible flexible support and stem cells and
n′) identifying a second upper spinal root and a second lower spinal root branching off from the spinal cord from a same second side, right or left, opposite to the first side, above and below the lesion in the spinal cord, respectively,
wherein said method comprises performing steps o1), o2), o3) and o4) also on said second upper and lower spinal roots, that is performing a bypass operation on both sides with respect to the spinal cord.

12. The surgical method of claim 9, wherein steps c1), c2) provide for cutting said upper and lower spinal roots and said second upper and lower spinal roots cleanly, without tearing and without bipolar coagulation.

13. The surgical method of claim 11, wherein steps o1), o2) provide for cutting said upper and lower spinal roots and said second upper and lower spinal roots cleanly, without tearing and without bipolar coagulation.

14. A surgical method for repairing a lesion in a spinal cord, comprising:
a) providing a medical device for repairing a lesion in a spinal cord or a peripheral nerve, said medical device comprising a biocompatible flexible support suitable for taking an extended configuration and a wound configuration and comprising stem cells suitable for being oriented along a first growth direction or a second growth direction,
wherein, in said wound configuration, the biocompatible flexible support is windable around the spinal cord and/or a spinal root, or a peripheral nerve, so that said first and second growth directions are substantially statistically parallel to a neuronal extension direction of neurons of the spinal cord or of the peripheral nerve,
wherein said biocompatible flexible support comprises an inner surface such that when the medical device is wound around said spinal cord and/or the spinal root or the peripheral nerve, the inner surface faces the spinal cord and/or the spinal root or said peripheral nerve, and
wherein the biocompatible flexible support is made of expanded polytetrafluoroethylene (ePTFE) and the stem cells are at least partially embedded on the inner surface of said biocompatible flexible support,
or manufacturing a medical device by a manufacturing method comprising
providing a biocompatible flexible support made of expanded polytetrafluoroethylene (ePTFE);
arranging stem cells on an inner surface of the biocompatible flexible support; and
depositing a growth factor on said inner surface to promote extension of said stem cells along a first growth direction or a second growth direction; and
b) winding said spinal cord by the medical device at the lesion in the spinal cord.

15. The surgical method of claim 8, wherein the medical device and/or the further medical device is a medical device comprising a biocompatible flexible support suitable for taking an extended configuration and a wound configuration and comprising stem cells suitable for being oriented along a first growth direction or a second growth direction,
wherein, in said wound configuration, the biocompatible flexible support is windable around the spinal cord and/or a spinal root, or a peripheral nerve, so that said first and second growth directions are substantially statistically parallel to a neuronal extension direction of neurons of the spinal cord or of the peripheral nerve,
wherein said biocompatible flexible support comprises an inner surface so that when the medical device is wound around said spinal cord and/or the spinal root or the peripheral nerve, the inner surface faces the spinal cord and/or the spinal root or said peripheral nerve, and
wherein the biocompatible flexible support is made of expanded polytetrafluoroethylene (ePTFE) and the stem cells are at least partially embedded on the inner surface of said biocompatible flexible support,
and/or the medical device is manufactured by a method comprising:
providing a biocompatible flexible support made of expanded polytetrafluoroethylene (ePTFE);
arranging stem cells on an inner surface of the biocompatible flexible support; and
depositing a growth factor on said inner surface to promote extension of said stem cells along a first growth direction or a second growth direction.

16. The surgical method of claim 11, wherein the medical device and/or the further medical device is a medical device comprising a biocompatible flexible support suitable for taking an extended configuration and a wound configuration and comprising stem cells suitable for being oriented along a first growth direction or a second growth direction,
wherein, in said wound configuration, the biocompatible flexible support is windable around the spinal cord and/or a spinal root, or a peripheral nerve, so that said first and second growth directions are substantially statistically parallel to a neuronal extension direction of neurons of the spinal cord or of the peripheral nerve,
wherein said biocompatible flexible support comprises an inner surface so that when the medical device is wound around said spinal cord and/or the spinal root or the peripheral nerve, the inner surface faces the spinal cord and/or the spinal root or said peripheral nerve, and
wherein the biocompatible flexible support is made of expanded polytetrafluoroethylene (ePTFE) and the stem cells are at least partially embedded on the inner surface of said biocompatible flexible support,
and/or the medical device is manufactured by a method comprising
providing a biocompatible flexible support made of expanded polytetrafluoroethylene (ePTFE);
arranging stem cells on an inner surface of the biocompatible flexible support; and
depositing a growth factor on said inner surface to promote extension of said stem cells along a first growth direction or a second growth direction.

17. The surgical method of claim 8, wherein the spinal cord comprises an outer layer of dura mater, or dural sac, an inner layer of pia mater and an intermediate layer of arachnoid interposed between the inner layer of pia mater and the dural sac,
wherein said surgical method comprises, before step b), cutting the dural sac and the intermediate layer of arachnoid, through which direct access to the inner layer of pia mater is allowed, from outside.

18. The surgical method of claim 14, wherein the spinal cord comprises an outer layer of dura mater, or dural sac, an inner layer of pia mater and an intermediate layer of arachnoid interposed between the inner layer of pia mater and the dural sac,
wherein said surgical method comprises, before step b), cutting the dural sac and the intermediate layer of arachnoid, through which direct access to the inner layer of pia mater is allowed, from outside.
